(19) United States
(12) Patent Application Publication
(10) Pub. No.: US 2023/0131417 A1
(43) Pub. Date: Apr. 27, 2023

(54) DEVICE TO MONITOR AND ALARM MANUAL VENTILATION PARAMETERS DURING CARDIOPULMONARY RESUSCITATION

(71) Applicant: Rush University Medical Center, Chicago, IL (US)

(72) Inventors: Jonathan Brady SCOTT, Wheaton, IL (US); Jie LI, Forest Park, IL (US)

(73) Assignee: Rush University Medical Center, Chicago, IL (US)

(21) Appl. No.: 17/800,503

(22) PCT Filed: Feb. 16, 2021
(86) PCT No.: PCT/US2021/018140

§ 371 Date: Aug. 17, 2022

Related U.S. Application Data
(60) Provisional application No. 62/977,804, filed on Feb. 18, 2020.

(51) Int. Cl. A61M 16/00; A61M 16/20

(52) U.S. Cl. CPC A61M 16/0084; A61M 16/024; A61M 16/0051; A61M 16/208; A61M 2016/0042; A61M 2205/50; A61M 2205/581; A61M 2205/583; A61M 2230/42; A61M 2230/432

(57) ABSTRACT

The disclosure is directed to an apparatus and a method for improving manual ventilation and resuscitation by monitoring ventilation parameters and assisting resuscitation. The apparatus includes a gas flow sensor configured to measure a flow rate of exhaled gas of a subject. The apparatus is configured to receive an ideal body weight or a predicated body weight of the subject and calculate a first tidal volume range based on the ideal body weight or the predicated body weight of the subject. The apparatus is also configured to obtain an exhaled tidal volume of the subject based on the measured flow rate and determine whether the exhaled tidal volume is within the first tidal volume range. When it is determined that the exhaled tidal volume is not within the first tidal volume range, the apparatus is further configured to perform a first tidal volume warning.

### RELATED APPLICATIONS

This application claims the benefit of the filing date of U.S. Provisional Pat. Application No. 62/977,804, filed Feb. 18, 2020, the contents of which is hereby incorporated by reference.

### BACKGROUND

### 1. Technical Field

This disclosure relates to a device and a method for improving manual ventilation and resuscitation, and particularly for monitoring manual ventilation parameters and assisting manual resuscitation.

### 2. Background Information

In an emergent situation, for example, during an accident or a surgery in hospital settings, a subject may become unresponsive with no breathing or breathing only in occasional agonal gasps. Cardiopulmonary resuscitation (CPR) may be administrated by a user with a manual resuscitator to treat the subject.

In 2016, the incidence of out-of-hospital cardiac arrest in the United States is more than 350,000. The currently available manual resuscitators lack functions of monitoring important ventilation parameters and assisting users to use the resuscitators properly. For example, the currently available manual resuscitators do not provide accurate measurements of the volume of gas disposed into the subject for each respiratory cycle, and do not provide assistance to the users with appropriate tidal volumes based on the needs of individual subjects. When tidal volumes are larger than the appropriate volume, lung injury may occur; when tidal volumes are smaller than the appropriate volume, hypoventilation may occur. Consequently, the usage of the currently available manual resuscitators may lead to reduction in cardiac output caused by lung over-distension or may lead to hypercapnia due to hypoventilation.

The present disclosure describes a device and a method for monitoring manual ventilation parameters and assisting manual resuscitation, and addresses at least some of the drawbacks listed above.

### BRIEF SUMMARY

The present disclosure describes an apparatus for improving manual ventilation and resuscitation. The apparatus includes a memory storing instructions, a processor in communication with the memory, and a gas flow sensor in communication with the processor. The flow sensor is configured to measure a flow rate of exhaled gas of a subject. When the processor executes the instructions, the instructions are configured to cause the apparatus to receive an ideal body weight or a predicated body weight of the subject and calculate a first tidal volume range based on the ideal body weight or the predicated body weight of the subject. When the processor executes the instructions, the instructions are configured to cause the apparatus to obtain an exhaled tidal volume of the subject based on the measured flow rate and determine whether the exhaled tidal volume is within the first tidal volume range. When the processor executes the instructions, the instructions are further configured to cause the apparatus to, when it is determined that the exhaled tidal volume is not within the first tidal volume range, perform a first tidal volume warning.

The present disclosure is also directed to a method for improving manual ventilation and resuscitation. The method includes receiving, by an apparatus, an ideal body weight or a predicated body weight of a subject. The apparatus includes a memory storing instructions, a processor in communication with the memory, and a gas flow sensor in communication with the processor. The gas flow sensor is configured to measure a flow rate of exhaled gas of the subject. The method includes calculating, by the apparatus, a first tidal volume range based on the ideal body weight or the predicated body weight of the subject. The method also includes obtaining, by the apparatus, an exhaled tidal volume of the subject based on the measured flow rate, and determining, by the apparatus, whether the exhaled tidal volume is within the first tidal volume range. The method further includes, when it is determined that the exhaled tidal volume is not within the first tidal volume range, performing, by the apparatus, a first tidal volume warning.

### BRIEF DESCRIPTION OF THE DRAWINGS

FIG. 1 is a schematic diagram of a system for manual resuscitation.

FIG. 2 is a schematic diagram of an apparatus for improving manual ventilation and resuscitation.

FIGS. 3A-3C are flow diagrams of a method for providing improving manual ventilation and resuscitation.

FIGS. 4A-4D are schematic diagrams of several embodiments of an input device of the apparatus for improving manual ventilation and resuscitation.

FIG. 5 is a diagram of the first and second tidal volume ranges.

FIG. 6 is a flow diagram of a method for monitoring a respiratory rate.

FIG. 7 is a flow diagram of a method for monitoring an end-tidal carbon dioxide.

FIG. 8 is a flow diagram of a method for assisting users with a consistent respiratory rate.

### DETAILED DESCRIPTION OF THE DISCLOSURE

The invention will now be described in detail hereinafter with reference to the accompanied drawings, which form a part of the present invention, and which show, by way of illustration, specific examples of embodiments. Please note that the invention may, however, be embodied in a variety of different forms and, therefore, the covered or claimed subject matter is intended to be construed as not being limited to any of the embodiments to be set forth below. Please also note that the invention may be embodied as methods, devices, components, or systems. Accordingly, embodiments of the invention may, for example, take the form of hardware, software, firmware or any combination thereof.

Throughout the specification and claims, terms may have nuanced meanings suggested or implied in context beyond an explicitly stated meaning. Likewise, the phrase “in one embodiment” or “in one implementation” as used herein does not necessarily refer to the same embodiment or implementation and the phrase “in another embodiment” or “in another implementation” as used herein does not necessarily refer to a different embodiment or implementation. It is intended, for example, that claimed subject matter includes combinations of exemplary embodiments or implementations in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure describes an apparatus and a method for monitoring manual ventilation parameters and assisting manual resuscitation. The apparatus may help users of a resuscitator with providing ventilation information of a subject during cardiopulmonary resuscitation (CPR), based on subject’s individual needs. The users of the resuscitator may include any health care providers, for example but not limited to, clinicians, physicians and nurses. The users of the resuscitator may also include other persons who are not health care providers, for example but not limited to, ordinary home users.

The ventilation information of the subject may include manual ventilation parameters, which may include one or more of the following: exhaled tidal volumes, respiratory rate, and an end-tidal carbon dioxide (CO2) pressure.

The apparatus may monitor manual ventilation parameters, determine whether each manual ventilation parameter is within a pre-determined range of the manual ventilation parameter, alert/alarm the user when the manual ventilation parameter is not within the pre-determined range. The alerting/alarming device for ventilation parameters may include one or more of the following: an audio message from a speaker, a light indicator of a certain color, and a symbol/text/picture displayed on a display.

The apparatus may include a timer on respiratory rate to remind the users to keep a consistent respiratory rate. The timer device may include one or more of the following: an audio message from a speaker, a blinking light of variable rates, and a symbol/text/picture displayed on a display.

The apparatus may be manufactured as either a disposable device or a reusable device. The apparatus may be portable.

The apparatus may include a storage component having the ability to store the ventilation parameters, and the stored ventilation parameters may be downloaded at a later time. The functions of storing and downloading ventilation parameters may increase research/quality improvement potentials.

The present disclosure may be better compliance with the American Heart Association recommendation of respiratory rate during CPR with an artificial airway in place. By monitoring tidal volumes, the present disclosure may help to get a more accurate tidal volume during CPR. Accurate tidal volumes during CPR may reduce lung injury caused by large tidal volumes or avoid hypoventilation by small tidal volumes. Appropriate respiratory rates and tidal volumes play important roles during CPR. The present disclosure may help to avoid the reduction in cardiac output caused by lung over distension.

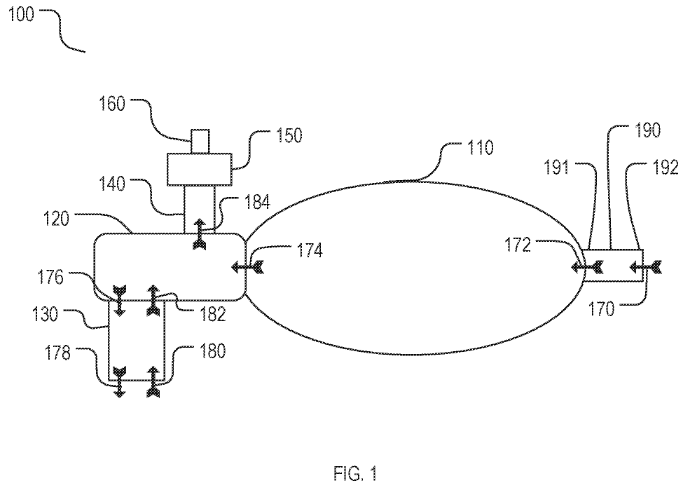

The present disclosure describes an embodiment of a system 100 for assisting manual resuscitation, as shown in FIG. 1. The system 100 may include a resuscitator bag 110, an intermediate coupler 120, an inspiratory coupler 130, an expiratory coupler 140, a monitoring/assisting device 150, a pressure-relief valve 160, and a gas inlet 190.

The resuscitator bag 110 may be a bag containing a certain volume of a gas such as air, oxygen, or a mixture of air and oxygen. When the resuscitator bag is compressed, it may provide a positive pressure ventilation to the subject. The resuscitator bag 110 may be made of flexible materials, for example but not limited to, silicone and polyvinyl chloride (PVC).

The intermediate coupler 120 may be a coupler connecting the resuscitator bag 110, the inspiratory coupler 130, and the expiratory coupler 140. The intermediate coupler 120 may include a valve assembly including one or more valves. The valve assembly is configured to have the gas flowing along directions as described below.

The inspiratory coupler 130 may provide an interface to the subject. The inspiratory coupler 130 may include one of an endotracheal (ET) tube and a face/mouth mask, which is configured to provide the gas to lungs of the subject.

The expiratory coupler 140 is configured to have exhaled gas passing through.

The monitoring/assisting device 150 may include one or more sensors to measure ventilation parameters, a processor to process the measurements from the sensors, alarm/alert users when a ventilation parameter is not within a pre-determined range, and assist users with proper resuscitation. The monitoring/assisting device 150 may fit and be disposed on an exhalation side of the system 100 to reduce secretion contamination on one or more sensors inside the monitoring/assisting device 150. The monitoring/assisting device 150 may be any one or a combination of any part of embodiments described below.

The pressure-relief valve 160 is a safety feature and may prevent high pressures being delivered inadvertently to the subject. The pressure-relief valve 160 is configured to release gas pressure when the gas pressure is above a preset threshold.

The gas inlet 190 may include a one-way valve, which is configured to allow gas drawn into the resuscitator bag 110. A first end 191 of the gas inlet 190 may be coupled with the resuscitator bag 110. A second end 192 of the gas inlet 190 may include one or more configurations, for example but not limited to, the second end 192 is exposed in air so that environmental air is drawn into the resuscitator bag; the second end 192 is configured to connect to a tube connecting to an oxygen reservoir so that oxygen gas is drawn into the resuscitator bag; the second end 192 is configured to connect to an oxygen intake assembly so that a mixture of air and oxygen at a per-determined mixture ratio is drawn into the resuscitator bag.

The system 100 may include one or more valves configured to ensure proper directions of gas flow. The gas is configured to be drawn into the resuscitator bag 110 with gas flowing along directions 170 and 172. During inspiratory cycle, the gas inside the resuscitator bag 110 may be compressed into the subject with gas flowing along directions 174, 176, and 178. During expiratory cycle, the exhaled gas from the subject may be exhaled with gas flowing along directions 180, 182, and 184.

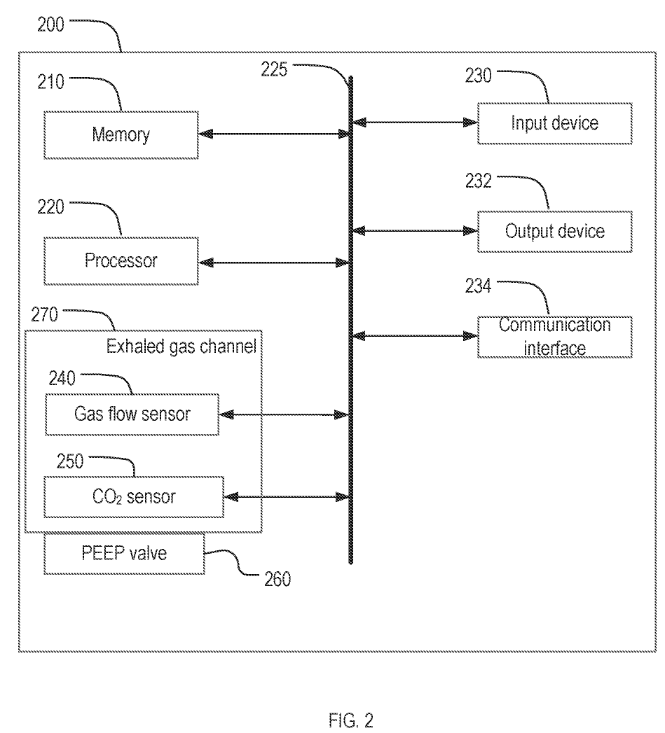

The present disclosure describes a monitoring/assisting device for monitoring manual ventilation parameters and assisting manual resuscitation. FIG. 2 is an exemplary structural diagram of a device 200. The device 200 may be one embodiment of the monitoring/assisting device 150 in FIG. 1.

The device 200 may include a memory 210, a processor 220, a bus 225, an input device 230, an output device 232, a communications interface 234, and a gas flow sensor 240. The device 200 may include other components, which are not shown in FIG. 2, for example but not limited to, a power source or a battery. In another embodiment, the device 200 may be used with an automated resuscitation system for monitoring ventilation parameters and assisting resuscitation.

The bus 225 may include a communication channel used for transferring information between components of a computer system. The memory 210, the processor 220, the input device 230, the output device 232, the communications interface 234, and the gas flow sensor 240 may be connected to each other by using the bus 225.

The memory 210 may store a program used for performing the technical solutions of the present disclosure, and may also store an operating system and other service. Specifically, the program may include program code, and the program code includes a computer operation instruction. More specifically, the memory 210 may include a read-only memory (ROM), a static storage device of another type that can store static information and an instruction, a random access memory (RAM), a dynamic storage device of another type that can store information and an instruction, a magnetic disk memory, a flash memory, or the like. The memory 210 may also store ventilation parameters measured during resuscitations.

The processor 220 may be a general purpose processor, for example, a general purpose central processing unit (CPU), a network processor (NP), or a microprocessor, or may be an application-specific integrated circuit (ASIC), or one or more integrated circuits configured to control execution of a program in the solutions of the present disclosure, or may be a digital signal processor (DSP), an ASIC, a field programmable gate array (FPGA), or another programmable logic device, a discrete gate, a transistor logic device, or a discrete hardware component.

The input device 230 may include an apparatus that receives data and information input by a user, for example but not limited to, a keyboard, a voice input apparatus, and a touch screen.

The output device 232 may include an apparatus that allows outputting information to the user, for example but not limited to, a display screen, a speaker, one or more lights with one or more colors.

The communications interface 234 may include an apparatus that uses any transceiver, so as to communicate with another device or a communications network, via wired communications or wireless communications, for example but not limited to, Ethernet, a radio access network (RAN), a wireless local area network (WLAN), a Bluetooth communication, and a Wi-Fi communication.

The gas flow sensor 240 may be disposed in an exhaled gas channel 270 of the device 200. The gas flow sensor 240 may measure flow rates of the exhaled gas of the subject, and send the measured data of flow rates to the bus 225, either continuously in real-time or at a pre-determined time intervals. The processor may calculate tidal volumes based on the measured data of flow rates. The gas flow sensor 240 may operate by measuring speed of the gas flow with techniques such as ultrasonic transducers, or by measuring pressure difference in the exhaled channel.

Optionally in another implementation, the device 200 may include a CO2 sensor 250 disposed in the exhaled gas channel 270 to measure the end-tidal CO2. The CO2 sensor 250 may send the measured data of end-tidal CO2 to the bus 225, either continuously or at a pre-determined time intervals.

Optionally in another implementation, the device 200 may include a positive end-expiratory pressure (PEEP) valve 260 disposed at an end/terminal of the exhaled gas channel 270. Under normal circumstances without PEEP valve, the pressure in the lung at the end of expiration is equal to the atmospheric pressure. PEEP valve may provide additional pressure at the end of expiration to maintain pressure in the lung slightly above atmospheric pressure. Therefore, PEEP valve may increase the volume of gas remaining in the lungs at the end of expiration, which may lead to a decrease of the shunting of blood through the lungs and an improvement of gas exchange in the lungs of the subject. PEEP valve may operates by means of a mechanical impedance, usually a valve, within the PEEP.

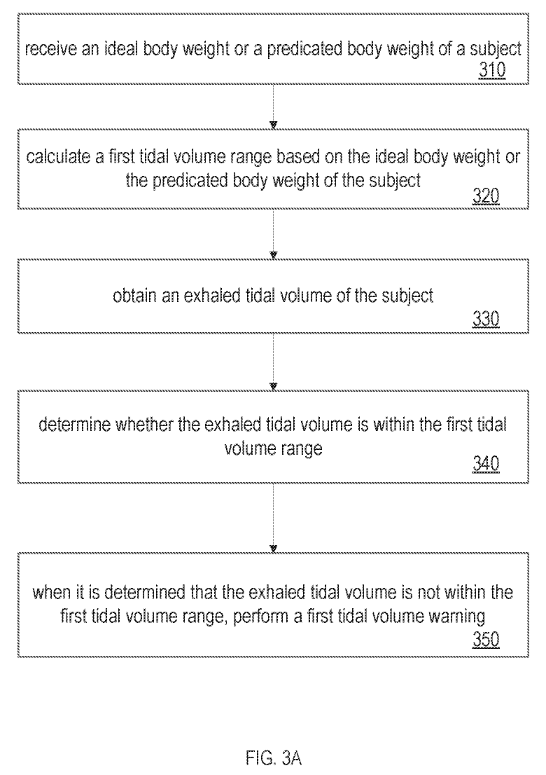

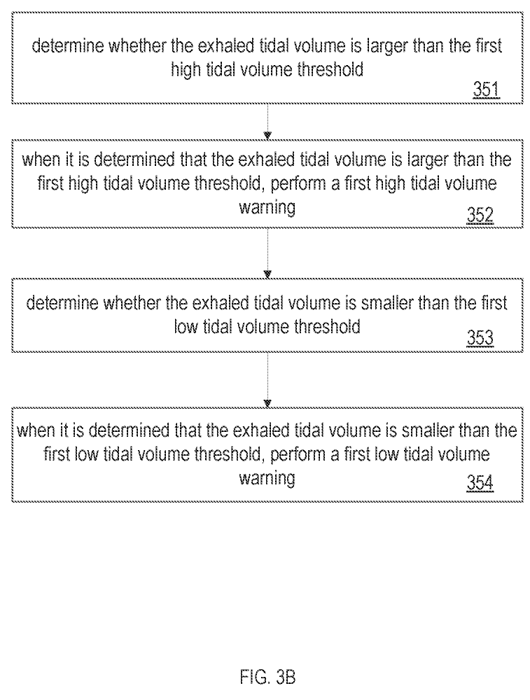

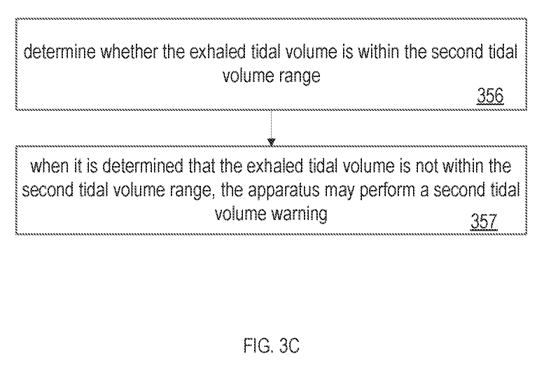

FIG. 3 shows an exemplary procedure of an apparatus for monitoring manual ventilation parameters and assisting manual resuscitation. The apparatus may include a memory storing instructions, a processor in communication with the memory, and a gas flow sensor in communication with the processor and configured to measure a gas flow rate of exhaled gas. When the processor executes the instructions, the instructions are configured to cause the apparatus to execute one or more of the following functions, as shown in FIGS. 3A-3C.

In 310, the apparatus may receive an ideal body weight or a predicted body weight of a subject. The apparatus may include an input device, which receives the ideal body weight or the predicted body weight.

An ideal body weight and a predicted body weight of a subject may be an effective weight and/or a calculated weight of the subject, which may depend on patient information of the subject. The ideal body weight and a predicted body weight may not be the same as an actual body weight of the subject. The patient information may include a height of the subject and/or a gender/sex of the subject. Optionally, the patient information may include an age of the subject.

In one implementation, users of the apparatus may obtain the ideal body weight or the predicted body weight of the subject based on the patient information. For example but not limited, the user may calculate the ideal body weight or the predicted body weight with a pre-determined formula based on the height and/or gender/sex of the subject; or the user may obtain the ideal body weight or the predicted body weight by using a look-up table. Depending on the age of the subject, the subject may be either an adult or a child, so that the user may use the appropriate per-determined formula or look-up table to determine the correct ideal body weight or predicted body weight. In another implementation, the input device of the apparatus may receive the patient information and the apparatus may obtain the ideal body weight or the predicted body weight of the subject based on the patient information. The pre-determined formula for calculating an ideal body weight may be: for male (Kg) = 50 + [0.9 x (Height (cm) – 154)]; and for female (Kg) = 45 + [0.9 * (Height (cm) – 154). The pre-determined formula for calculating a predicated body weight may be: for male (Kg) = 50 + 2.3 * (Height (in) – 60); and for female (Kg) = 45.5 + 2.3 * (height (in) -60). In another implementation, a predicted body weight based on a height and a gender/sex of a subject is shown in Table 1.

TABLE 1

Predicted body weight (PBW)

| Height (ft/inch) | PBW for female (Kg) | PBW for male (Kg) |
|---|---|---|
| 4′ 0″ | 17.9 | 22.4 |
| 4′ 1″ | 20.2 | 24.7 |
| 4′ 2″ | 22.5 | 27 |
| 4′ 3″ | 24.8 | 29.3 |
| 4′ 4″ | 27.1 | 31.6 |
| 4′ 5″ | 29.4 | 33.9 |
| 4′ 6″ | 31.7 | 36.2 |
| 4′ 7″ | 34 | 38.5 |
| 4′ 8″ | 36.3 | 40.8 |
| 4′ 9″ | 38.6 | 43.1 |
| 4′ 10″ | 40.9 | 45.4 |
| 4′ 11″ | 43.2 | 47.7 |
| 5′ 0″ | 45.5 | 50 |
| 5′ 1″ | 47.8 | 52.3 |
| 5′ 2″ | 50.1 | 54.6 |
| 5′ 3″ | 52.4 | 56.9 |
| 5′ 4″ | 54.7 | 59.2 |
| 5′ 5″ | 57 | 61.5 |
| 5′ 6″ | 59.3 | 63.8 |
| 5′ 7″ | 61.6 | 66.1 |
| 5′ 8″ | 63.9 | 68.4 |
| 5′ 9″ | 66.2 | 70.7 |
| 5′ 10″ | 68.5 | 73 |
| 5′ 11″ | 70.8 | 75.3 |
| 6′ 0″ | 73.1 | 77.6 |
| 6′ 1″ | 75.4 | 79.9 |
| 6′ 2″ | 77.7 | 82.2 |
| 6′ 3″ | 80 | 84.5 |
| 6′ 4″ | 82.3 | 86.8 |
| 6′ 5″ | 84.6 | 89.1 |
| 6′ 6″ | 86.9 | 91.4 |
| 6′ 7″ | 89.2 | 93.7 |
| 6′ 8″ | 91.5 | 96 |
| 6′ 9″ | 93.8 | 98.3 |
| 6′ 10″ | 96.1 | 100.6 |
| 6′ 11″ | 98.4 | 102.9 |
| 7′ 0″ | 100.7 | 105.2 |

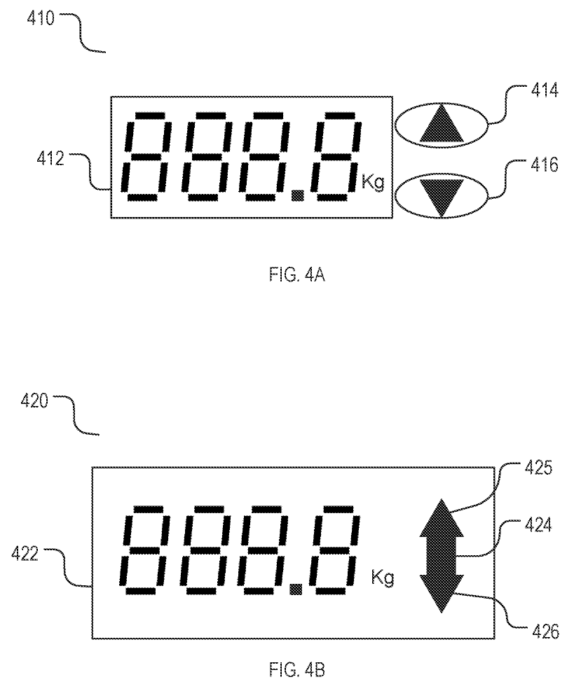

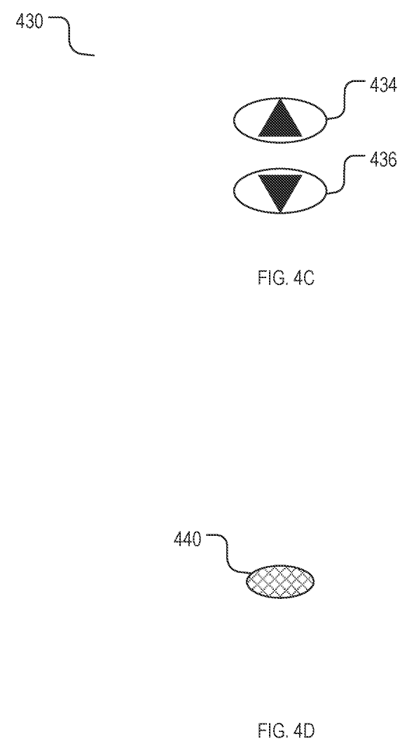

FIGS. 4A-4D described a few exemplary input devices to receive the ideal body weight or the predicated body weight of the subject.

For the below disclosure, the ideal body weight may be used as an example. The described method/device may be applied similarly to the predicted body weight as well.

In one implementation, the input device may be a device 410 shown in FIG. 4A. The device 410 may include a digital display 412 to display an ideal body weight of a subject. The ideal body weight may be displayed in different units, for example but not limited to, kilogram (Kg) and pound (lb). The digital display 412 may be a seven-segment display for displaying decimal numerals.

The device may include a plurality of buttons, for example, an increase button 414 and a decrease button 416. A user of the device may press the increase and/or decrease buttons to increase/decrease the ideal body weight of the subject so that the display 412 displays the correct ideal body weight of the subject.

In another implementation, the input device may be a device 420 as shown in FIG. 4B. The device 420 may include a touch screen display 422. The ideal body weight of the subject may be displayed on one portion of the touch screen display 422. A sliding symbol 424 may be displayed on one portion of the touch screen to adjust the ideal body weight. A touch/click of the top portion 425 of the sliding symbol may increase the ideal body weight, and a touch/click of the bottom 426 of the sliding symbol may decrease the ideal body weight.

In another implementation, the input device may be a device 430 as shown in FIG. 4C. The device may include an increase button 434 and a decrease button 436. When a user of the device may press the increase buttons, an ideal body weight of the subject stored in the memory may increase by a fixed amount and a speaker of the apparatus may announce the ideal body weight to inform the user. When a user of the device may press the decrease buttons, the ideal body weight of the subject stored in the memory may decrease by the fixed amount and the speaker of the apparatus may announce the current ideal body weight to inform the user.

In another implementation, the input device may be a microphone 440 as shown in FIG. 4D. A user of may speak the ideal body weight of the subject, and the microphone 440 receives the voice and the apparatus may obtain the ideal body weight by a voice-to-text process. The apparatus may use a speaker to announce the obtained ideal body weight to ask the user to confirm whether the obtained ideal body weight is correct. Consequently, the voice of user’s confirmation may be received by the microphone and processed by the apparatus by the voice-to-text process.

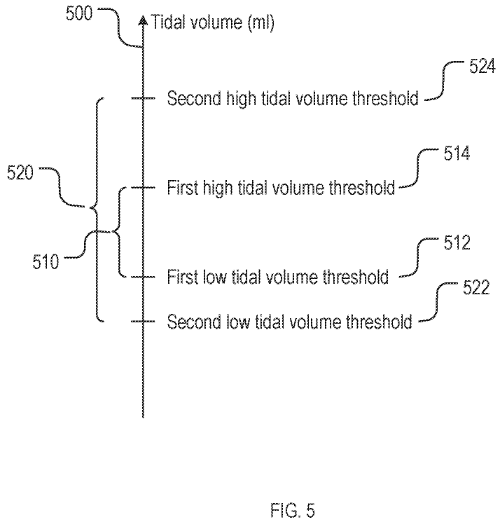

In 320, the apparatus may calculate a first tidal volume range based on the ideal body weight of the subject. The first tidal volume range 510 along a tidal volume axis 500 may be any value being smaller than a first high tidal volume threshold 514 and larger than a first low tidal volume threshold 512, as shown in FIG. 5. The first high tidal volume threshold and the first low tidal volume threshold may be calculated based on a pre-determined formula. For example but not limited to, the first high tidal volume (milliliter, ml) threshold may be the ideal body weight (in Kg) multiplied by 8 ml/Kg; and the first low tidal volume threshold may be the ideal body weight (in Kg) multiplied by 6 ml/Kg.

In another implementation, the apparatus may calculate a second tidal volume range based on the ideal body weight of the subject. The second tidal volume range 520 may be any value being smaller than a second high tidal volume threshold 524 and larger than a second low tidal volume threshold 522. The second high tidal volume threshold and the second low tidal volume threshold may be calculated based on a pre-determined formula. For example but not limited to, the second high tidal volume (milliliter, ml) threshold may be the ideal body weight (in Kg) multiplied by 10 ml/Kg; and the second low tidal volume threshold may be the ideal body weight (in Kg) multiplied by 5 ml/Kg.

In 330, the apparatus may obtain an exhaled tidal volume of the subject. The gas flow sensor of the apparatus may be disposed in an exhaled gas channel in the apparatus. The gas flow sensor may measure flow rates of the exhaled gas of the subject, and send the measured data of flow rates to the processor. The processor may obtain tidal volumes based on the measured flow rates for one respiratory cycle. In one implementation, the apparatus may inform the obtained exhaled tidal volume to the users by a text-to-voice from a speaker or a display of the obtained exhaled tidal volume on the display.

In 340, the apparatus may determine whether the exhaled tidal volume is within the first tidal volume range. In 350, when it is determined that the exhaled tidal volume is not within the first tidal volume range, the apparatus may perform a first tidal volume warning.

The first tidal volume range may be considered as a “normal” range for the subject. Thus, when the exhaled tidal volume is not within the first tidal volume range, the apparatus may alert/alarm the user the situation so that the use may take actions to correct the issue.

The first tidal volume warning may include a first tidal volume warning audio message, and/or a first tidal volume warning visual message. For example but not limited to, the first tidal volume warning audio message may include a beep sound from a speaker, and/or a either pre-recorded or machine-generated text-to-voice; the first tidal volume warning visual message may include a warning light, a flashing light, a flashing symbol on the display, and a warning text/symbol/graphic message on the display.

In one embodiment, the apparatus may perform different warning messages depending on whether the exhaled tidal volume is larger or smaller than the “normal” range. In one implementation, in 351, the apparatus may determine whether the exhaled tidal volume is larger than the first high tidal volume threshold. In 352, when it is determined that the exhaled tidal volume is larger than the first high tidal volume threshold, the apparatus may perform a first high tidal volume warning. In 353, the apparatus may determine whether the exhaled tidal volume is smaller than the first low tidal volume threshold. In 354, when it is determined that the exhaled tidal volume is smaller than the first low tidal volume threshold, the apparatus may perform a first low tidal volume warning. The first low and high tidal volume warnings may differ by one or more of: a pitch/duration of the beep sound, voice content, a color of the warning light, a flashing rate of the flashing light, and a color/shape of symbols displayed on the display. For example but not limited to, the first low tidal volume warning may include a lower pitch of the beep sound than the first high tidal volume warning, or the first low tidal volume warning may include a red color of light and the first high tidal volume warning may include a blue color of light.

In one implementation, the apparatus may assist a user with using a manual resuscitator. The first low tidal volume warning may inform the user that the present tidal volume is smaller than the “normal” range, so that the user may take actions to increase the volume of gas feeding into lungs of the subject; the first high tidal volume warning may inform the user that the present tidal volume is larger than the “normal” range, so that the user may take actions to decrease the volume of gas feeding into lungs of the subject. For example but not limited to, the actions may include a change of the force/extent of compressing a resuscitator bag of the manual resuscitator. An increase of the force/extent of compressing the resuscitator bag may increase the tidal volume, and a decrease of the force/extent of compressing the resuscitator bag may decrease the tidal volume.

In another implementation, the apparatus assists may assist a user with using an automated resuscitation system. The first low tidal volume warning may inform the user that the present tidal volume is smaller than the “normal” range, so that the user and/or the automated resuscitation system may take actions to increase the volume of gas feeding into lungs of the subject; the first high tidal volume warning may inform the user that the present tidal volume is larger than the “normal” range, so that the user and/or the automated resuscitation system may take actions to decrease the volume of gas feeding into lungs of the subj ect.

In another embodiment, the apparatus may determine whether the exhaled tidal volume is out of the second tidal volume range, and when the exhaled tidal volume is out of the second tidal volume range, perform a second tidal volume warning, which may be different from the first tidal volume warning. In 356, the apparatus may determine whether the exhaled tidal volume is within the second tidal volume range. In 357, when it is determined that the exhaled tidal volume is not within the second tidal volume range, the apparatus may perform a second tidal volume warning.

In one implementation, when the exhaled tidal volume is not within the second tidal volume range, the exhaled tidal volume is further off the “normal” range, and the second tidal volume warning may be different from the first tidal volume warning, for example but not limited to, higher pitch/ longer duration of the beep sound, voice with serious content, a more reddish color of the warning light, a higher flashing rate of the flashing light, and serious/dramatic symbol color/shape on the display.

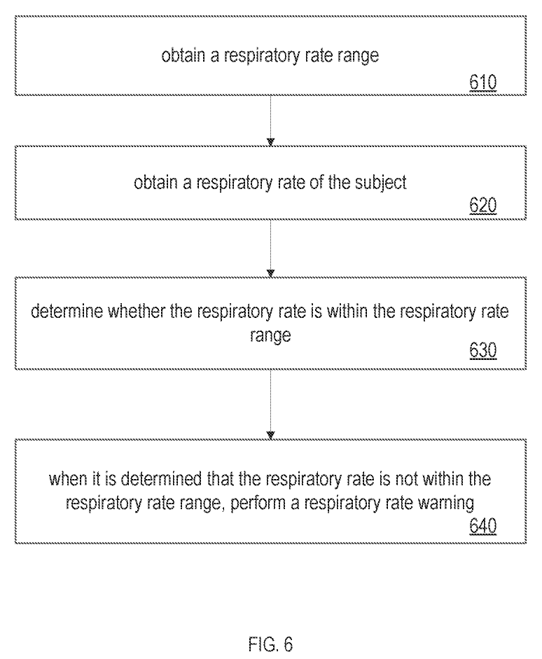

In another embodiment, the ventilation parameters may include a respiratory rate. The respiratory rate is the number of respiratory cycles of the subject in one minute. When the processor executes the instructions, the instructions are configured to cause the apparatus to execute one or more of the following functions, as shown in FIG. 6.

In 610, the apparatus may obtain a respiratory rate range. The respiratory rate range may be any value larger than a low respiratory rate threshold and smaller than a high respiratory rate threshold. The low respiratory rate threshold may be any number lower than 10, for example but not limited to, 5 for most subjects except small children. The high respiratory rate threshold may be any number larger than 10, for example but not limited to, 20 for most subjects except small children. The low and high respiratory rate thresholds may be different for small children.

In 620, the apparatus may obtain a respiratory rate of the subject. The inspiratory and expiratory period of the respiratory cycle may be obtained by analyzing the flow rate measured by the gas flow sensor. In one implementation, the apparatus may obtain how many respiratory cycles in one minute based on the fluctuation of the flow rate measured by the gas flow sensor. The flow rate measured by the gas flow sensor is larger during expiratory period than during inspiratory cycle. In another implementation, the apparatus may measure a time interval between successive respiratory cycles and then calculate the respiratory rate based on the time interval.

In another embodiment, the apparatus may include a respiratory sensor to measure the respiratory rate. The respiratory sensor may send the measured respiratory rate to the processor for processing.

In 630, the apparatus may determine whether the respiratory rate is within the respiratory rate range. In 640, when it is determined that the respiratory rate is not within the respiratory rate range, the apparatus may perform a respiratory rate warning.

Similar to the embodiment discussed above for the exhaled tilde volume, the apparatus may perform different warnings depending on whether the respiratory rate is larger than the high respiratory rate threshold or smaller than the low respiratory rate threshold.

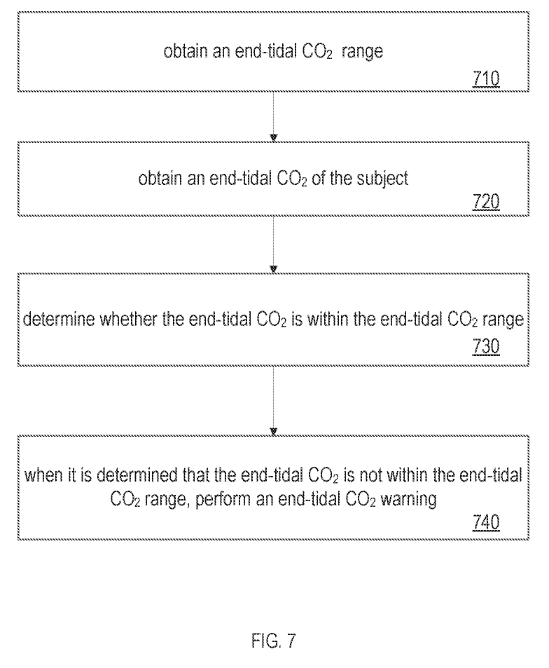

In another embodiment, the ventilation parameters may include an end-tidal CO2. The end-tidal CO2 may be used to access the quality of CPR and to monitor for the return of spontaneous circulation (ROSC). A measured end-tidal CO2 may be a partial pressure of CO2 in a unit of millimeter mercury (mmHg). When the processor executes the instructions, the instructions are configured to cause the apparatus to execute one or more of the following functions, as shown in FIG. 7.

In 710, the apparatus may obtain an end-tidal CO2 range. The end-tidal CO2 range may be any value larger than a low end-tidal CO2 threshold and smaller than a high end-tidal CO2 threshold. The low end-tidal CO2 threshold may be any value lower than 35 mmHg, for example 20 mmHg for most subjects except small children. The high end-tidal CO2 threshold may be any number larger than 35 mmHg, for example 45 mmHg for most subjects except small children.

In 720, the apparatus may obtain an end-tidal CO2 of the subject. The apparatus may include a CO2 sensor to measure the end-tidal CO2. The CO2 sensor may send the measured end-tidal CO2 to the processor for processing.

In 730, the apparatus may determine whether the end-tidal CO2 is within the end-tidal CO2 range. In 740, when it is determined that the end-tidal CO2 is not within the end-tidal CO2 range, the apparatus may perform an end-tidal CO2 warning.

Similar to the embodiment discussed above for the exhaled tilde volume, the apparatus may perform different warnings depending on whether the end-tidal CO2 is larger than the high end-tidal CO2 threshold the or smaller than the low end-tidal CO2 threshold.

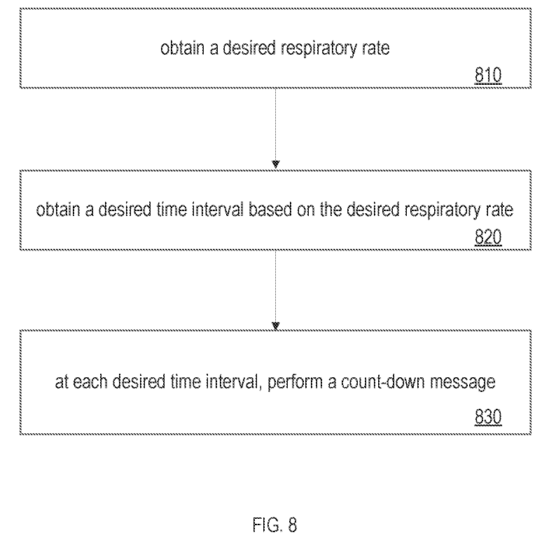

In another embodiment, FIG. 8 shows an exemplary procedure of an apparatus for assisting users of the apparatus to keep a respiratory rate consistent.

In 810, the apparatus may obtain a desired respiratory rate. The desired respiratory rate may be any value determined by health care providers, for example but not limited to, 10 per minute for most subjects except small children.

In 820, the apparatus may obtain a desired time interval based on the desired respiratory rate. When the desired respiratory rate is 10 per minute, the desired time interval between successive respiratory cycles is 6 seconds (60 seconds/10 = 6 seconds).

In 830, at each desired time interval, the apparatus may perform a count-down message. The count-down message at each desired time interval may inform the user on the timing of allowing gas into the lungs of the subject, for example, by compressing a resuscitator bag of a manual resuscitator. When the desired respiratory rate is 10 per minute, the apparatus may perform a count-down message every 6 seconds to assist the users to keep a consistent respiratory rate.

The count-down message may include an audio message and/or a visual message. For example but not limited to, the audio message may include a beep sound from a speaker, whose pitch be higher as the court-down progresses to zero; the visual message may include a flashing light, whose flashing rate may be larger as the court-down progresses to zero.

The court-down message may include a number displayed on a display. For example but not limited to, the display may be a seven-segment digital display for displaying decimal numerals. Therefore, when the desired respiratory rate is 10 per minute, the digital display may count down from 6 to 0 at a rate of one number per second, and the count-down may repeat every 6 seconds.

While the particular invention has been described with reference to illustrative embodiments, this description is not meant to be limiting. Various modifications of the illustrative embodiments and additional embodiments of the invention will be apparent to one of ordinary skill in the art from this description. Those skilled in the art will readily recognize that these and various other modifications can be made to the exemplary embodiments, illustrated and described herein, without departing from the spirit and scope of the present invention. It is therefore contemplated that the appended claims will cover any such modifications and alternate embodiments. Certain proportions within the illustrations may be exaggerated, while other proportions may be minimized. Accordingly, the disclosure and the figures are to be regarded as illustrative rather than restrictive.

1. An apparatus for improving manual ventilation and resuscitation, the apparatus comprising:
a memory storing instructions;
a processor in communication with the memory;
a gas flow sensor in communication with the processor, wherein the gas flow sensor is configured to measure a flow rate of exhaled gas of a subject; and
wherein, when the processor executes the instructions, the instructions are configured to cause the apparatus to:
receive an ideal body weight or a predicated body weight of the subject;
calculate a first tidal volume range based on the ideal body weight or the predicated body weight of the subject;
obtain an exhaled tidal volume of the subject based on the measured flow rate;
determine whether the exhaled tidal volume is within the first tidal volume range; and
when it is determined that the exhaled tidal volume is not within the first tidal volume range, perform a first tidal volume warning.

2. The apparatus according to claim 1, wherein, when the instructions are configured to cause the apparatus to receive the ideal body weight or the predicated body weight of the subject, the instructions are configured to cause the apparatus to:
receive patient information of the subject, wherein the patient information comprises a height and a gender of the subject; and
obtain the ideal body weight or the predicated body weight of the subject based on the patient information.

3. The apparatus according to claim 1, further comprising:
a carbon dioxide sensor in communication with the processor, wherein the carbon dioxide sensor is configured to measure an end-tidal carbon dioxide of the subject; and
wherein, when the processor executes the instructions, the instructions are configured to cause the apparatus to:
obtain an end-tidal carbon dioxide range;
obtain the end-tidal carbon dioxide of the subject;
determine whether the end-tidal carbon dioxide is within the end-tidal carbon dioxide range; and
when it is determined that the end-tidal carbon dioxide is not within the end-tidal carbon dioxide range, perform an end-tidal carbon dioxide warning.

4. The apparatus according to claim 3, wherein
the end-tidal carbon dioxide range comprises a range being larger than 20 mmHg and smaller than 45 mmHg.

5. The apparatus according to claim 1, wherein, when the processor executes the instructions, the instructions are further configured to cause the apparatus to:
obtain a respiratory rate range;
obtain a respiratory rate of the subject;
determine whether the respiratory rate is within the respiratory rate range; and
when it is determined that the respiratory rate is not within the respiratory rate range, perform a respiratory rate warning.

6. The apparatus according to claim 5, wherein
the respiratory rate range comprises a range being larger than 5 per minute and smaller than 20 per minute.

7. The apparatus according to claim 1, wherein, when the processor executes the instructions, the instructions are further configured to cause the apparatus to:
obtain a desired respiratory rate;
obtain a desired time interval based on the desired respiratory rate; and
at each desired time interval, perform a count-down message.

8. The apparatus according to claim 7, wherein:
the desired respiratory rate is 10 per minute.

9. The apparatus according to claim 1, further comprising:
a positive end-expiratory pressure (PEEP) valve.

10. The apparatus according to claim 1, wherein:
the first tidal volume warning comprises at least one of an audio message and a visual message.

11. The apparatus according to claim 10, wherein:
the audio message comprises at least one of:
a beep sound by a speaker of the apparatus,
a pre-recorded voice by the speaker of the apparatus, and
a text-to-voice generated by the apparatus; and
the visual message comprises at least one of:
light from a warning light of the apparatus,
flashing light of the warning light,
a flashing symbol displayed on a display of the apparatus, and
a warning message displayed on the display, the warning message comprising at least one of a text, a symbol, or a graphic message.

12. The apparatus according to claim 1, wherein:
the first tidal volume range comprising a range being larger than a first low tidal volume threshold and smaller than a first high tidal volume threshold; and
when the instructions are configured to cause the apparatus to calculate the first tidal volume range based on the ideal body weight or the predicated body weight of the subject, the instructions are configured to cause the apparatus to:
multiply the ideal body weight or the predicated body weight and 8 ml/Kg to obtain the first high tidal volume threshold, and
multiply the ideal body weight or the predicated body weight and 6 ml/Kg to obtain the first low tidal volume threshold.

13. The apparatus according to claim 12, wherein, when the processor executes the instructions, the instructions are further configured to cause the apparatus to:
determine whether the exhaled tidal volume is larger than the first high tidal volume threshold;
when it is determined that the exhaled tidal volume is larger than the first high tidal volume threshold, perform a first high tidal volume warning;
determine whether the exhaled tidal volume is smaller than the first low tidal volume threshold; and
when it is determined that the exhaled tidal volume is smaller than the first low tidal volume threshold, perform a first low tidal volume warning.

14. The apparatus according to claim 1, wherein, when the processor executes the instructions, the instructions are further configured to cause the apparatus to:
calculate a second tidal volume range based on the ideal body weight or the predicated body weight of the subject;
determine whether the exhaled tidal volume is within the second tidal volume range; and
when it is determined that the exhaled tidal volume is not within the second tidal volume range, perform a second tidal volume warning.

15. The apparatus according to claim 14, wherein:
the second tidal volume warning is different from the first tidal volume warning.

16. The apparatus according to claim 1, wherein, when the processor executes the instructions, the instructions are further configured to cause the apparatus to:
store the exhaled tidal volume of the subject; and
send the stored exhaled tidal volume to a second device.

17. The apparatus according to claim 1, wherein:
the apparatus is disposed at an exhalation side of a manual resuscitator.

18. A method for improving manual ventilation and resuscitation, the method comprising:
receiving, by an apparatus, an ideal body weight or a predicated body weight of a subject, wherein the apparatus comprises a memory storing instructions, a processor in communication with the memory, and a gas flow sensor in communication with the processor and configured to measure a flow rate of exhaled gas of the subject;
calculating, by the apparatus, a first tidal volume range based on the ideal body weight or the predicated body weight of the subject;
obtaining, by the apparatus, an exhaled tidal volume of the subject based on the measured flow rate;
determining, by the apparatus, whether the exhaled tidal volume is within the first tidal volume range; and
when it is determined that the exhaled tidal volume is not within the first tidal volume range, performing, by the apparatus, a first tidal volume warning.

19. The method according to claim 18, wherein:
the apparatus further comprising a carbon dioxide sensor in communication with the processor, wherein the carbon dioxide sensor is configured to measure an end-tidal carbon dioxide of the subject; and
the method further comprises:
obtaining, by the apparatus, an end-tidal carbon dioxide range;
obtaining, by the apparatus, the end-tidal carbon dioxide of the subject;
determining, by the apparatus, whether the end-tidal carbon dioxide is within the end-tidal carbon dioxide range; and
when it is determined that the end-tidal carbon dioxide is not within the end-tidal carbon dioxide range, performing, by the apparatus, an end-tidal carbon dioxide warning.

20. The method according to claim 18, further comprising:
obtaining, by the apparatus, a respiratory rate range;
obtaining, by the apparatus, a respiratory rate of the subject;
determining, by the apparatus, whether the respiratory rate is within the respiratory rate range; and
when it is determined that the respiratory rate is not within the respiratory rate range, performing, by the apparatus, a respiratory rate warning.
